(19) United States
(12) Patent Application Publication
(10) Pub. No.: US 2008/0210540 A1
(43) Pub. Date: Sep. 4, 2008

(54) SEPARATION AND DEWATERING OF ORGANIC SOLVENTS BY INTEGRATING DISTILLATION AND MEMBRANE SEPARATION OPERATIONS

(76) Inventor: Rex A. Dieterle, Madison, WI (US)

Correspondence Address: MICHAEL BEST & FRIEDRICH LLP, ONE SOUTH PINCKNEY STREET, P O BOX 1806, MADISON, WI, 53701, US

(21) Appl. No.: 12/015,902

(22) Filed: Jan. 17, 2008

Related U.S. Application Data
(60) Provisional application No. 60/885,300, filed on Jan. 17, 2007.

(51) Int. Cl. C07C 29/80

(52) U.S. Cl. 203/19

(57) ABSTRACT

The present invention provides a system and a process for the recovery of an organic solvent from a mixture of an organic solvent and water. The process comprises subjecting an organic solvent/water mixture to a distillation system whereby a vapor is produced. The output from the distillation process passes through a filtration unit which comprises a hydrophilic membrane filter. The solvent/water mixture passing through the filtration unit is separated into a concentrate and a permeate. The concentrate (containing mostly organic solvent) can be collected, can be routed to the distillation system for further purification, or can be routed to a second filtration system for further purification. The permeate (containing mostly water) is routed from the filtration unit to a condenser where it is condensed and collected.

### CROSS REFERENCE TO RELATED APPLICATIONS

This application claims the benefit of priority from U.S. Provisional Patent Application No. 60/885,300 filed Jan. 17, 2007, the application incorporated herein by reference in its entirety.

### FIELD OF THE INVENTION

The present invention relates to processes for recovery of an organic solvent or solvents from a mixture of the solvent(s) and water. The compounds are separated by means of a pervaporation system which is integrated with a fractional distillation system.

Normal distillation operations require the recycle of high amounts of the finished product back to the distillation operation called reflux which normally is up to 3 to 10 times the amount of the product produced to produce a final dry product. This requires large amounts of energy to condense and then evaporate the same material over and over again.

One industry that currently suffers from the challenges of organic solvent/water separation is the ethanol industry. Ethanol is produced from the conversion of sugars to ethanol and CO₂ by yeast fermentation in a water solution. Corn based ethanol plants convert the corn starch to sugar before fermentation but other waste sugar processes convert the sugars directly, as in the cheese whey permeate process where lactose sugar is converted to ethanol. Because the yeasts are sensitive to high concentrations of ethanol, the fermentation beer product will generally range from less than 5% ethanol to as high as 18-20% ethanol. The typical ethanol water concentration is 8-12% ethanol.

To separate the ethanol from water, the “beer” recovered from the ethanol fermentation process is distilled. Commercial continuous packed columns or plate stills are used. The ethanol water mixture is heated and enters the distillation column near the center. The beer is then continuously condensed and then evaporated as the water moves down the column and is taken out of the system and the ethanol moves up the column as dried product. To achieve a high purity of product at the top of the distillation column, an amount of the final product is recycled back to the distillation column in the range of ⅔ to 9/10 of the actual product recovered. Because of the need for recycle or reflux of the product to achieve high product purity, it takes about twice as much energy to produce a 95% ethanol product as an 85% ethanol product.

With ethanol distillation there is an additional problem in that, at atmospheric pressures, the best you can do is produce a 95% ethanol product. This is due to the production of an azeotrope in the distillation process. Once the azeotrope is produced, no amount of extra energy will separate the remaining 5% of the water out. Originally a third solvent was distilled with the ethanol water to break the azeotrope. This method was extremely energy intense. Now the accepted method is to use a molecular sieve or to operate the distillation column in a vacuum or a combination of both.

In the molecular sieve method, ethanol water vapor is passed over a packed bed of zeolite crystals which remove the water. Nanometer holes in the crystals trap the water as the ethanol passes. While this method is very efficient in trapping the water, to regenerate the crystals a large portion of the dried product must be super heated and then recycled back through the beds and then back to the distillation column for recovery. This requires additional energy use for the super heating of the dried product. This method requires complex automation and control equipment and involves operating at temperatures with ethanol vapor in the 350° to 450° F. range.

In the vacuum method, the azeotrope can be broken by operating the distillation column at a 1/10 atmosphere. The overall temperature of the distillation column is lower but requires a high reflux ratio of 10 to 20. This high number of refluxes requires the use of large amounts of energy. The energy requirement for pure distillation with no azeotrope is 1.4 times the energy of the combustion energy of ethanol. Most corn ethanol distillations now are done at a slight vacuum which requires higher distillation energy per gallon then atmospheric or pressure distillation.

Given the problems with current separation techniques, a more energy efficient system and process for separating organic solvents from solution in a more energy efficient fashion is needed.

### SUMMARY OF THE INVENTION

The present invention provides a system and a process for the recovery of an organic solvent from a mixture of an organic solvent and water.

In one embodiment, the process comprises subjecting an organic solvent/water mixture to a fractional distillation column whereby a vapor is produced. Vapors from the distillation process are condensed into a liquid. The liquid is then passed through a filtration unit which comprises a hydrophilic membrane filter. The liquid passing through the filtration unit is separated into a concentrate (retentate) and a permeate. The concentrate (containing mostly organic solvent) can be collected, can be routed to the fractional distillation column for further purification, or can be routed to a second filtration unit for further purification. The permeate (containing mostly water) is routed from the filtration unit to a condenser where it is condensed and collected.

In another embodiment, the process comprises subjecting an organic solvent/water mixture to a fractional distillation column whereby a vapor is produced. Vapors from the distillation are passed through a filtration unit which comprises a hydrophilic membrane filter. The vapor passing through the filtration unit is separated into a concentrate (retentate) and a permeate. The concentrate (containing mostly organic solvent) can be collected, can be routed to the fractional distillation column, or can be routed to a second filtration system for further purification. The permeate (containing mostly water) is routed from the filtration unit to a condenser where it is condensed and collected.

In another embodiment, the invention comprises a separation system. The separation system comprises a fractional distillation column; at least a first filtration unit (additional filtration units can be used), and a retentate collection tank.

The fractional distillation column can be of any known design. Suitably the fractional distillation column comprises a housing having a lower section, a middle section and an upper section. A primary product input feed and a reflux input line are connected to the middle section of the housing. An overhead vapor output line is connected to the upper section of the housing. In addition, a reboiler input and output can be connected to the lower section of the housing. The lower section of the distillation housing can also have a water input line.

The filtration unit comprises a membrane filter, a feed input line, a concentrate output line and a permeate output line. A vacuum pump is operatively connected to the permeate output line of the membrane filtration unit.

The overhead vapor output line of the fractional distillation column is operatively connected to the feed input line of the filtration unit. These connected lines may be optionally interspersed with a pump or pumps for moving the vapors from the distillation column to the filtration unit. The separation system can also contain a condenser for condensing the vapors recovered from the distillation column into a liquid, a recovery tank for the condensed liquid, and/or heat exchanging unit(s) for regulating the temperature of the vapors or condensed liquid recovered from the distillation column before they enter the first filtration unit.

The concentrate output line of the filtration unit can be operatively connected to a product collection tank, the reflux input line of the fractional distillation column, and to a recycle loop back to the feed input line of the filtration unit. Each of these lines contains a valve, suitably an automated valve, so that an operator can direct the flow of the concentrate to the collection tank (for the collection of the concentrate), the fractional distillation column (for re-distillation and further purification of the concentrate), or back into the input of the filtration unit (for further filtration and purification of the concentrate). Optionally, the concentrate output line can be operatively connected to an input line of an additional filtration unit. These connected lines may be optionally interspersed with a pump or pumps for moving the concentrate from the filtration unit to either the concentrate collection tank, the fractional distillation column, a second filtration unit, or back to the input of the filtration unit that the concentrate is coming out of. Additionally these lines may optionally contain heat exchanging unit(s) for regulating the temperature of the concentrate.

The permeate output line of the filtration unit can be operatively connected to a condenser unit which condenses the permeate into a liquid. The condenser unit can also be operatively connected by an output line to a permeate recovery tank, in which the permeate liquid is recovered. These connected lines may be optionally interspersed with a pump or pumps for moving the permeate from the filtration unit, to the condenser, and from the condenser to the recovery tank.

### BRIEF DESCRIPTION OF THE DRAWINGS

FIG. 1 is a systems diagram showing one embodiment of the system of the present invention.

FIG. 2 is a diagram showing liquid permeation of a membrane filter.

FIG. 3 is a diagram of a pervaporation process.

FIG. 4 is a schematic of one embodiment of the separation system of the present invention wherein the solvent/water mixture is processed in a liquid form.

FIG. 5 is a schematic of one embodiment of the separation system of the present invention wherein the solvent/water mixture is processed in a vapor form.

FIG. 6 is a schematic of one embodiment of the separation system of the present invention wherein the solvent/water mixture undergoes successive filtrations.

Before the embodiments of the invention are explained in detail, it is to be understood that the invention is not limited in its application to the details of construction and the arrangements of the components set forth in the following description or illustrated in the drawings. The invention is capable of other embodiments and of being practiced or being carried out in various ways. Also, it is understood that the phraseology and terminology used herein are for the purpose of description and should not be regarded as limiting. The use of “including”, “having” and “comprising” and variations thereof herein is meant to encompass the items listed thereafter and equivalents thereof as well as additional items and equivalents thereof.

It also is understood that any numerical value recited herein includes all values from the lower value to the upper value. For example, if a concentration range is stated as 1% to 50% , it is intended that values such as 2% to 40% , 10% to 30% , or 1% to 3% , etc., are expressly enumerated in this specification. These are only examples of what is specifically intended, and all possible combinations of numerical values between the lowest value and the highest value enumerated are to be considered to be expressly stated in this application.

### DETAILED DESCRIPTION OF THE INVENTION

The present invention provides a system and a process for the recovery of an organic solvent from a mixture of an organic solvent and water. The compounds are separated by means a pervaporation system which is integrated to a fractional distillation system.

Pervaporation is an energy efficient combination of membrane permeation and evaporation. Pervaportion separation uses low temperatures and pressures and has cost and performance advantages for the separation of constant-boiling azeotropes. Pervaporation is useful for the separation of heat sensitive products, for diluting solutions containing trace amounts of a component to be used, and for the dehydration of organic solvents and the removal of organics from aqueous streams.

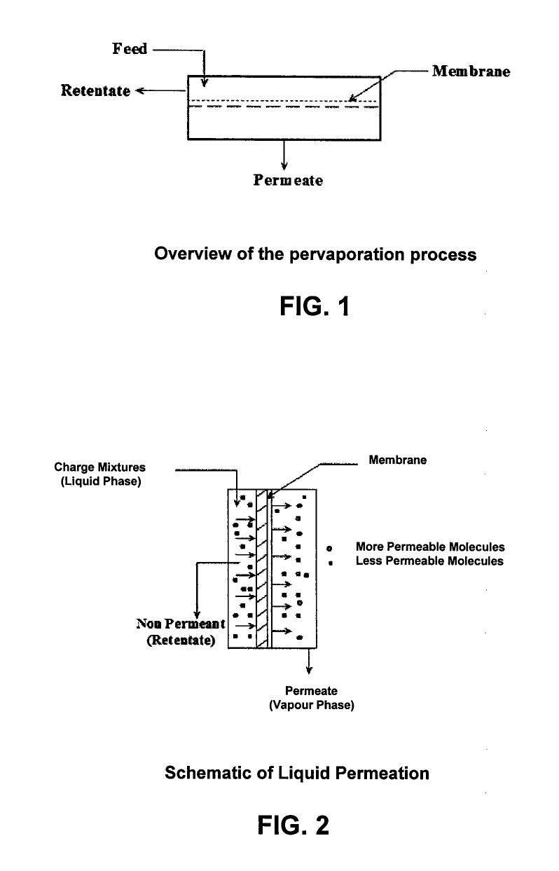

Pervaporation involves the separation of two or more components across a membrane by differing rates of diffusion through a thin polymer and an evaporative phase change comparable to a simple flash step. A concentrate and vapor pressure gradient is used to allow one component to preferentially permeate across the membrane. A vacuum applied to the permeate side is coupled with the condensation of the permeated vapors. Pervaporation is typically suited to separating a minor component of a liquid mixture, thus high selectivity through the membrane is essential. FIG. 1 shows an overview of the pervaporation process. FIG. 2 shows a schematic of liquid permeation in a pervaporation system.

Liquid transport in pervaporation is described by various solution-diffusion models. The steps included are the sorption of the permeate at the interface of the solution feed and the membrane, diffusion across the membrane due to concentration gradients (rate determining steps), and finally desorption into a vapor phase at the permeate side of the membrane. The first two steps are primarily responsible for the permselectivity. As material passes through the membrane a “swelling” effect makes the membrane more permeable, but less selective, until a point of unacceptable selectivity is reached and the membrane must be regenerated.

The other driving force for separation is the difference in partial pressures across the membrane. By reducing the pressure on the permeate side of the membrane, a driving force is created. Another method of inducing a partial pressure gradient is to sweep an inert gas over the permeate side of the membrane. These methods are described as vacuum and sweep gas pervaporation respectively.

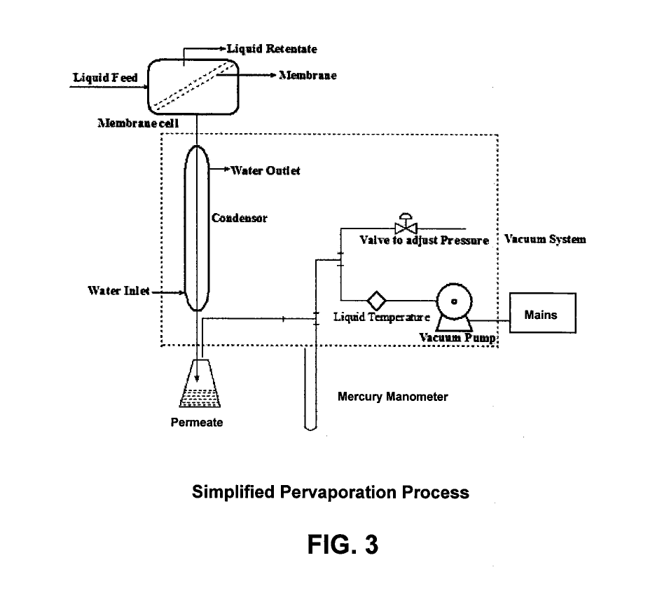

A basic pervaporation system is shown in FIG. 3. In this embodiment, the filtration unit comprises a membrane cell having a membrane filter, a feed input line, a concentrate (retentate) output line and a permeate output line. A condensation unit is also present, the condensation unit having a housing, an input operatively connected to the permeate output line, an output line, a water inlet line and a water outlet line. A vacuum pump is operatively connected to the output line of the condensation unit. The mixture of organic solvents and water enter through the feed input line of the membrane cell and flow along one side of the membrane filter and a fraction of the feed (permeate) passes through the membrane and leaves in the vapor phase on the opposite side of the membrane. The “vapor phase” side of the membrane is kept under a vacuum. In another embodiment, the “vapor phase” side of the membrane can be purged with a stream of inert carrier gas. The permeate is finally collected in the liquid state after condensation. The liquid product is rich in the more rapidly permeating component of the feed mixture. The concentrate is made up of the feed materials that cannot pass through the membrane.

Hydrophilic membranes are used to remove water from organic solutions. These types of membranes are typically made of polymers with glass transition temperatures above room temperatures. Polyvinyl alcohol is an example of a hydrophilic membrane material. Organophilic membranes are used to recover organics from solutions. These membranes are typically made up of elastomer materials (polymers with glass transition temperatures below room temperature). The flexible nature of these polymers make them ideal for allowing organic materials to pass through. Examples include nitrile, butadiene rubber, and styrene butadiene rubber. Non-organic hydrophilic membranes based upon silicates, such as zeolites, may also be used. Zeolites can be created when nanometer thin layers of perm-selective coatings are applied to microporous ceramic support materials. Non-organic hydrophilic pervaporation membranes have the advantage of operation at higher temperatures and are more stable. Hydrophobic membranes are used to remove organic solvent from water solutions. These membranes can take the place of distillation, chromatography and other low organic concentration separation operations.

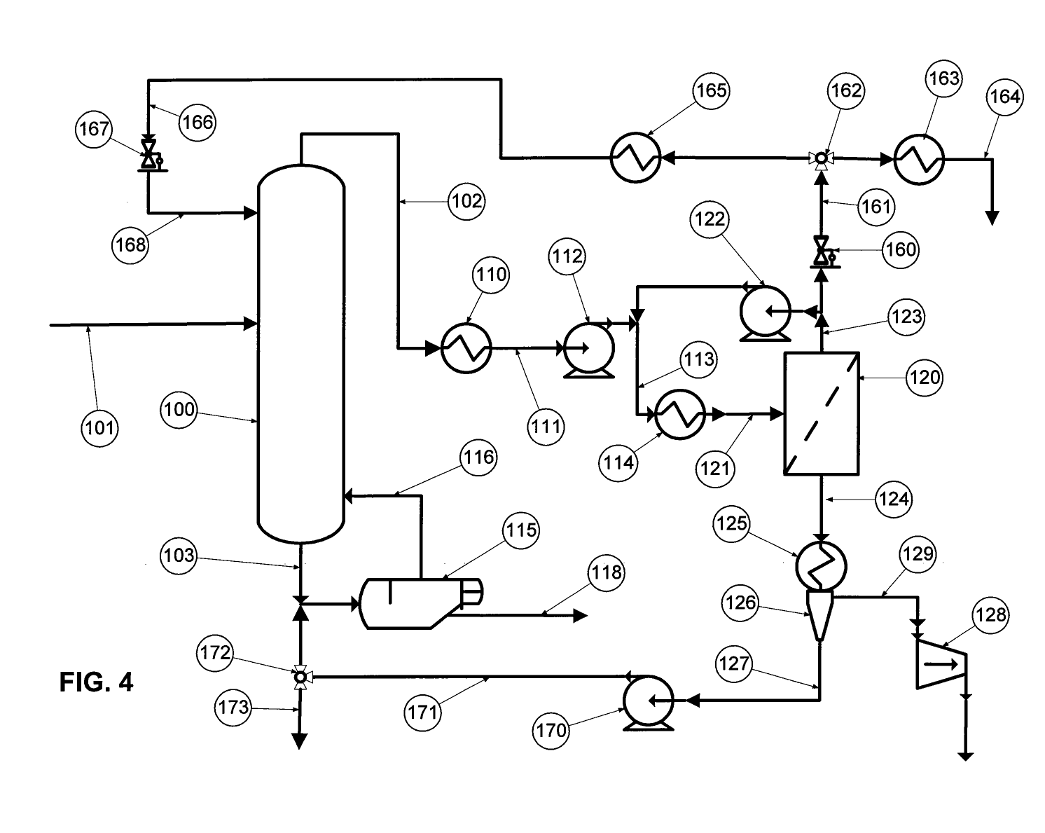

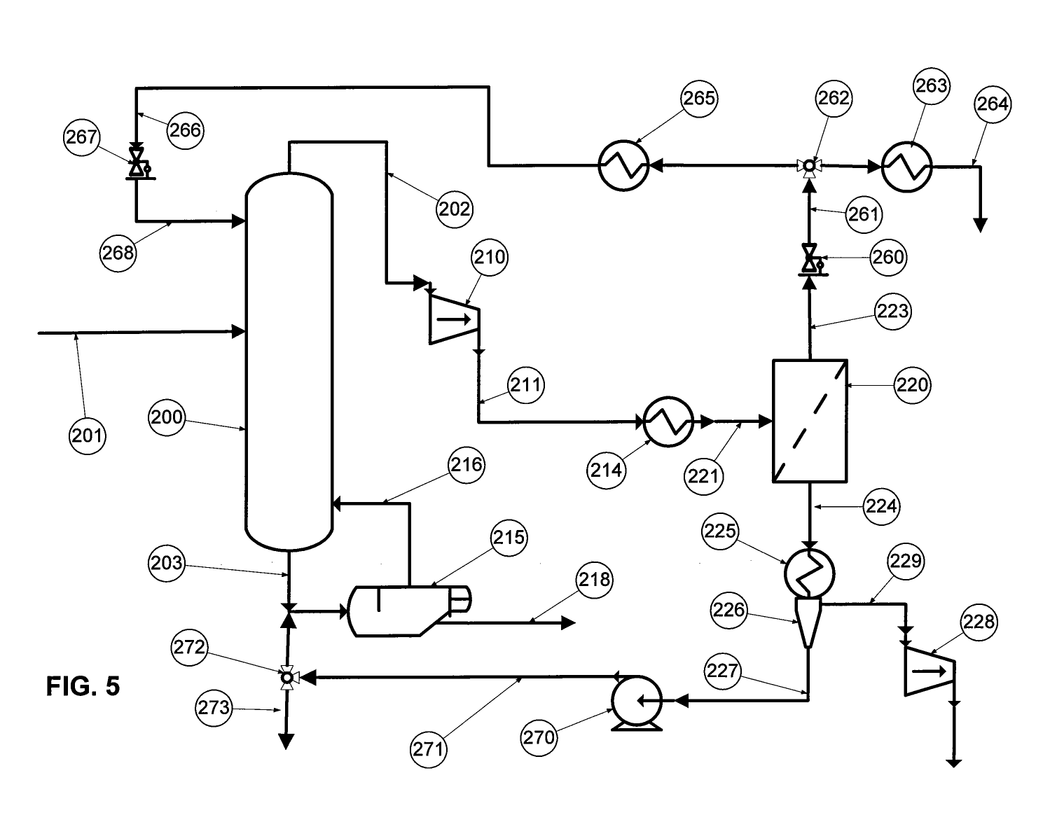

FIGS. 4-6 are schematic flow diagrams depicting the different embodiments of the integrated fractional distillation and separation system of the present invention. FIGS. 4 and 5 depict the process and apparatus of the invention for two separate embodiments for the simultaneous recovery of a very pure concentrate stream, an integrated permeate stream and one or more desired products from the distillation column. FIG. 4 depicts the operation of the system where the concentrate side of the system is kept in the liquid state. FIG. 5 depicts the operation of the system where the concentrate side of the system is kept in the gaseous state. Both systems are equally effective in separations of products but either one may be selected based upon ease of selection of process equipment, operation and/or the conservation of energy for each operation. Suitably, the liquid system may be selected for small flow rate and intermittent systems and the gaseous system for high flow rate continuous systems.

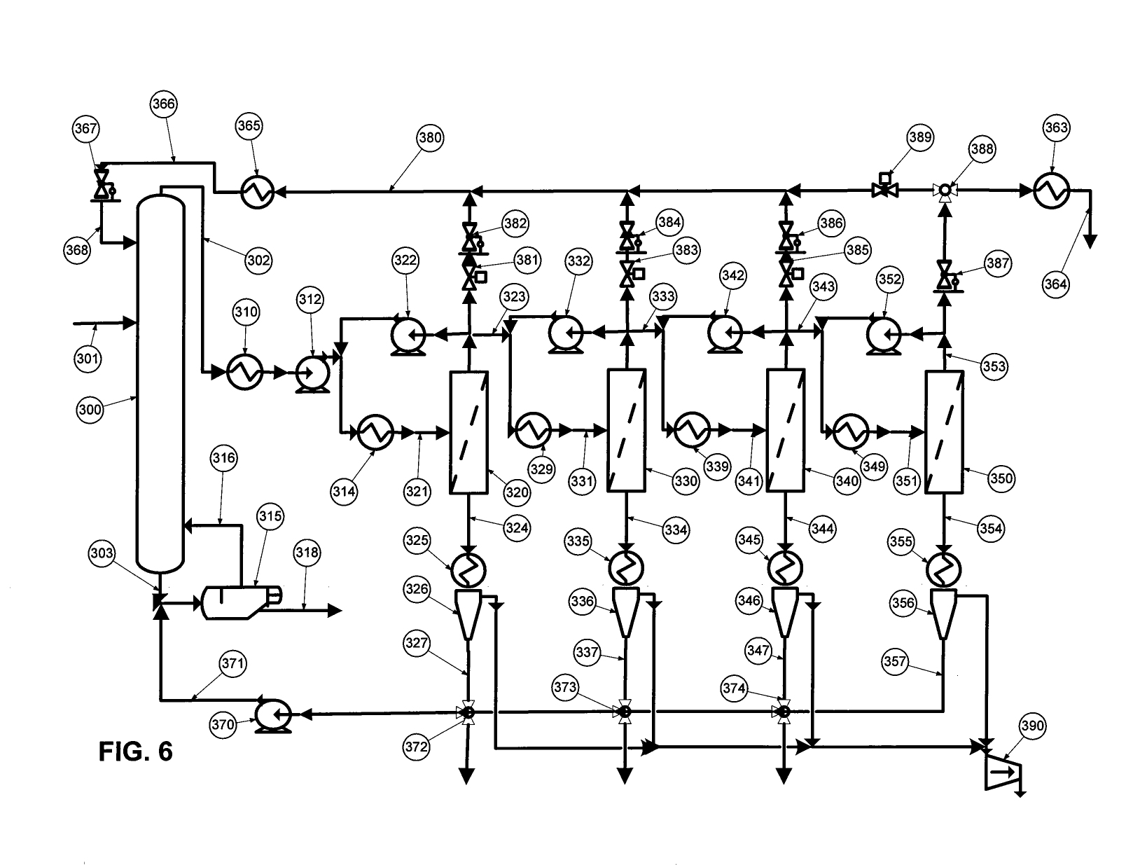

FIG. 6 describes a more specific configuration of the process utilizing multiple membrane filtration steps to provide process alternatives for optimizing the flow and recycle of products with the system. The liquid system is shown in FIG. 6, but the vapor system would operate in a similar manor.

Compounds that can be separated by the process and system of the present invention include, but are not limited to: alcohols (including methanol, ethanol, propanol, butanol (all isomers), pentanol, cyclohexanol and benzyl alcohol); aromatics (including benzene, toluene and phenol); esters (including methyl acetate, ethyl acetate, butyl acetate); organic acids (including acetic acid); ketones (including acetone, butanone and methyl isobutyl ketone); amines (including triethylamine, pyridine and aniline); aliphatics (including chlorinated hydrocarbons, dichloro methane and perchloroethylene; and ethers (including methyl tert-butyl ether (MTBE), ethyl tert-butyl ether (ETBE), di-isopropyl ether (DIPE), tetrahydro furan (THF) and dioxane).

### EXAMPLES

### Example 1

### Separation of Liquid Solvent/Water Mixture

FIG. 4 depicts a system for the separation of organic solvent from a liquid solvent/water mixture. The primary feed 101 delivers the solvent/water mixture into the fractional distillation column 100. This feed is normally positioned near the center of the column and conditioned for temperature and pressure based upon the overall optimal design of the distillation column which will take into consideration the quantity and quality of the membrane concentrate recycle reflux 168 described later. The overhead gaseous product 102 of the fractional distillation column 100 is directed to the filtration unit 120 which comprises a membrane filter. The overhead gaseous product 102 is condensed to a liquid 111 by passing the vapors through a condenser 110. The driving forces for the pervaporation separation is predominately the partial pressure difference between the concentrate side of the membrane and the permeate side of the membrane. To reduce the need for very low pressures and condensing temperatures on the permeate side of the membrane, higher pressures and temperatures may be selected for the concentrate side of the membrane. Suitably, temperatures can ran range from approximately 180 degrees Fahrenheit to approximately 300 degrees Fahrenheit and pressures can range from approximately 15 PSIG to approximately 80 PSIG. Liquid pump 112 routes the condensed solvent/water mixture 113 to heat exchanger 114. Heat exchanger 114 conditions the condensed solvent/water mixture to be an input feed 121 to the filtration unit 120. The pressure within the filtration unit 120 is held with the back pressure control valve 160. To make sure the flow within the concentrate side of the membrane is sufficient to minimize concentrating polarizations of the concentrate side of the membrane, a portion of the output concentrate 123 is routed to pump 122 and recycled back through heat exchanger 114 into the filtration unit 120 as an input 121.

The purified concentrate product 161 of the filtration unit 120 leaves the filtration unit 120 and is routed through control valve 162 where a portion of the flow 166 can be routed back to the fractional distillation column 100 as high quality reflux 168 and the remaining portion can leave the system as purified product 164. The purified product's 164 temperature is lowered to storage temperatures by heat exchanger 163 and passes out of the system. The recycle/reflux stream 168 can be furthered conditioned by lowering its temperature and pressure by heat exchanger 165 and pressure relief valve 167 to match the conditions of the distillation column 100 where it will be input.

The water or other small molecule gasses are separated out of the concentrate by passing through the filtration unit 120 in the vapor state. A vacuum is drawn on the permeate side of the membrane in filtration unit 120 by a vacuum pump 128 connected by conduit 129. Suitably, the vacuum operates in a range of approximately 500 Torr to approximately 0.5 Torr. To recover the permeate vapor 124 and not pass it through the vacuum pump 128, it is condensed by condenser 125 and collected in a gas liquid separator 126 where the vacuum is applied to the filtration unit 120. To aid in the collection of the permeate, the gas liquid separator will be attached to a barometric leg 127 at its bottom to produce a liquid height with its accompanying positive pressure at the leg bottom to reduce the suction pressure head that the permeate pump 170 must pull against. The condensed permeate at the bottom of the barometric leg 127 will be pumped through pump 170 to a flow direction valve 172 to exit the system as a product or waste water material 173.

The condensed permeate may still contain some level of organic product depending on the selectivity of the membrane filter. If full recovery of the product and the further purification of the permeate is desirable, the condensed permeate will be pumped through pump 170 and flow 171 is directed to the re-boiler 115 of the distillation column 100 by flow direction valve 172 and mixed with the distillation column 100 bottom product 103. The permeate will then be further purified and leave the system as the distillation column 100 bottom product 118. The small amount of organic solvent remaining in the permeate will re-enter the distillation column 100 as reheated bottoms product recycle 116 and will be re-captured.

### Example 2

### Separation of Gaseous Solvent/Water Mixture

FIG. 5 depicts a system for the separation of organic solvent from a gaseous solvent/water mixture. The primary feed 201 delivers the solvent/water mixture (either in a gaseous or liquid state) into a fractional distillation column 200. This feed is normally positioned near the center of the column and conditioned for temperature and pressure based upon the overall optimal design of the distillation column which will take into consideration the quantity and quality of the membrane concentrate recycle reflux 268 described later. The overhead gaseous product 202 of the distillation column 200 is directed to the filtration unit 220 which comprises a membrane filter. The overhead gaseous product 202 is compressed by compressor 210 and is delivered 211 to heat exchanger 214. The temperature and pressure of the gas is heated and compressed to increase the partial pressures of the gas to utilize higher pressures and temperatures on the pervaporation side of the membrane filter. The driving forces for the pervaporation separation is predominately the partial pressure difference between the concentrate side of the membrane filter and the permeate side of the membrane filter. To reduce the need for very low pressures and condensing temperatures on the permeate side of the membrane filter, higher pressures and temperatures may be selected for the concentrate side of the membrane filter. The conditioned gas 221 is the input feed to the filtration unit 220. Suitably temperatures are in the range of approximately 180 degrees Fahrenheit to approximately 300 degrees Fahrenheit and pressures are from approximately 15 PSIG to approximately 80 PSIG. The pressure within the filtration unit 220 is held with the back pressure control valve 260. Normally, the gaseous flow velocity on the concentrate side of the membrane does not have to be recycled as shown in FIG. 5, however, a recycled flow can be incorporated in the system if necessary.

The purified concentrate product leaves the filtration unit 220 through flows 223 and 261 to a control valve 262. A portion of the flow can be routed back to the distillation column 200 as high quality reflux 268 and the remaining portion can leave the system as purified product 264. The purified product's 264 temperature is lowered to storage temperatures by heat exchanger 263 and passes out of the system. The recycle/reflux stream 268 flows 266 from the control valve 262. The recycle/reflux stream 268 can be furthered conditioned by lowering its temperature and pressure by heat exchanger 265 and pressure relief valve 267 to match the conditions of the distillation column 200 where it will be input.

The water or other small molecule gases are separated out of the concentrate by passing through the membrane filter in the filtration unit 220 in the vapor state. A vacuum is drawn on the permeate side of the membrane filter by a vacuum pump 228 connected by conduit 229. Suitably, the vacuums operate in a range of approximately 500 Torr to approximately 0.5 Torr. To recover the permeate vapor 224 and not pass it through the vacuum pump 228, it is condensed by condenser 225 and collected in a gas liquid separator 226 where the vacuum is applied to the filtration unit 220. To aid in the collection of the permeate, the gas liquid separator will be attached to a barometric leg 227 at its bottom to produce a liquid height with its accompanying positive pressure at the leg bottom to reduce the suction pressure head that the permeate pump 270 must pull against.

The condensed permeate may still contain some level of organic product, depending on the selectivity of the membrane filter of the filtration unit 220. If full recovery of the product and the further purification of the permeate is desirable, the condensed permeate 227 will be pumped through pump 270 and flow 271 and is directed to the re-boiler 215 of the distillation column 200 by flow direction valve 272 and mixed with the distillation column 200 bottom product 203. The permeate will then be further purified and leave the system as the distillation column 200 bottom product 218. The small amount of organic solvent remaining in the permeate will re-enter the distillation column 200 as reheated bottoms product recycle 216 and will be re-captured.

### Example 3

### Separation of Ethanol from Ethanol/Water Beer Produced by Ethanol Fermentation.

FIG. 6 depicts a system for the separation of ethanol from an ethanol/water beer produced by ethanol fermentation. This is an example of one use of the invention that will improve the typical distillation and drying of fuel ethanol produced by fermentation of corn or other sugar substrates. Because of the energy efficiencies of the process and ease of operation, small distillation and drying plants of less than 1-5 MGY (Million Gallons per year) are practicable and would typically use the liquid system embodiment of the invention.

The ethanol/water mixture (fermentation beer) 301 suitably can come to the system at 5% to 20% ethanol by volume. The fermentation beer 301 is fed into fractional distillation column 300 through a primary feed. This feed is normally positioned near the center of the column and conditioned for temperature and pressure based upon the overall optimal design of the distillation column which will take into consideration the quantity and quality of the membrane concentrate recycle reflux 368 described later. In one embodiment, the distillation column 300 can be operated at a vacuum down to approximately 0.6 atmospheres or pressurized up to approximately 3 atmospheres of pressure. The factional distillation column 300 produces concentrated vapors 302 which are an ethanol enriched product. The concentrated vapors 302 leave the fractional distillation column through an overhead vapor outlet. The ethanol enriched concentrated vapors 302 are suitably in the 50% to 85% by volume range of organic solvent. In one embodiment, the ethanol enriched concentrated vapors 302 are condensed to a liquid by condenser 310 and pumped by pump 312 into the first filtration unit 320. In another embodiment, the ethanol enriched concentrated vapors 302 are introduced to the first filtration unit 320 in a vapor (gaseous) form. In one embodiment, the energy provided by pump 312 is sufficient to pump the product fluid through the entire system.

A trimming heat exchanger 314 can be used to fine adjust the temperature to the ethanol enriched product 302. Suitably, conditions for the ethanol enriched product 302 before it is introduced 321 to the first filtration unit 320 are temperatures that range from approximately 200F. to approximately 400F. and pressures from approximately 14 PSIG to approximately 80 PSIG. The pressure is kept within the system by a back pressure valve 387 at the output of the final filtration unit 350. For more complex systems, further control can be done by a back pressure valve at each filtration unit.

The ethanol enriched product 302 contacts the selective membrane of the first filtration unit 320, a portion of the first ethanol enriched product passes through the selective membrane, this portion is enriched in water and is a water permeate 324, and a second portion of the first ethanol enriched liquid being further enriched in ethanol not passing through the first selective membrane, this portion being a first ethanol concentrate 323. The separation process is the same for subsequent processing by additional filtration units (330, 340, 350).

The basic flow of the product through the system is to route the exit concentrate 323, 333, and 343 from one filtration unit 320, 330, and 340 through a trimming heat exchanger 314, 329, 339, and 349 to the next filtration unit 330, 340 and 350. The trimming heat exchangers 314, 329, 339, and 349 add the amount of heat that is lost due to the heat of evaporation within the stage as the water is evaporated through the membrane filter in the filtration units 320, 330, 340 and 350 and leaves the system so that the system can operate at a constant temperature in each stage. In other embodiments, these trimming heat exchanges can be eliminated and can have reduced temperatures at each succeeding stage.

The input product stream of the ethanol enriched product enters the first stage of filtration as input 321 to filtration unit 320. The concentrate 323 leaves the first filtration stage and is heated by heat exchanger 329 and enters the second filtration stage as input 331 to the second filtration unit 330. In some embodiments, a portion of the concentrate 323 can be routed to pump 322 and recycled back into the filtration unit 320 as an input 321.

The concentrate 333 leaves the second filtration stage and is heated by heat exchanger 339 and enters the third filtration stage as input 341 to the third filtration unit 340. In some embodiments, a portion of the concentrate 333 can be routed to pump 332 and recycled back into the filtration unit 330 as an input 331.

The concentrate 343 leaves the third filtration stage and is heated by heat exchanger 349 and enters the final filtration stage as input 351 to the final filtration unit 350. In some embodiments a portion of the concentrate 343 can be routed to pump 342 and recycled back into the filtration unit 340 as an input 341.

The final concentrated product 353 exits the system through the back pressure valve 387. The temperature of the concentrated product 353 can be dropped to storage temperature by heat exchanger 363 and be delivered to storage 364. In some embodiments, a portion of the concentrate 353 can be routed to pump 352 and recycled back into the filtration unit 350 as an input 351. If a portion of the final product 353 is selected to be recycled back to the distillation column 300 as reflux 368, divert valve 388 can divert a portion of the flow to the distillation column 300 controlled by control valve 389.

The water is removed from each stage by creating a vacuum on the permeate side of each filtration unit 320, 330, 340 and 350. Permeate vapors 324, 334, 344 and 354 are condensed by condensers 325, 336, 345 and 355 and separated by a liquid gas separator 326, 336, 346 and 356. A vacuum pump 390 provides the vacuum.

To aid in the collection of the permeate, the gas liquid separator will be attached to a barometric leg 327, 337, 347 and 357 at its bottom to produce a liquid height with its accompanying positive pressure at the leg bottom to reduce the suction pressure head that the permeate pump 370 must pull against.

The condensed permeate may still contain some level of organic product depending on the selectivity of the membrane filter of the filtration units 320, 330, 340 and 350. If full recovery of the product and the further purification of the permeate is desirable, the condensed permeate 327, 337, 347 and 357 can be pumped by pump 370 and flow 371 to the re-boiler 315 of the distillation column 300 and mix with the distillation column 300 bottom product 303. The permeate will then be further purified and leave the system as the distillation column 300 bottom product 318. The small amount of organic solvent remaining in the permeate will re-enter the distillation column 300 as reheated bottoms product recycle 316 and will be re-captured.

If the purity of the condensed permeate 327, 337, 347 and 357 is high enough, it can be removed from the system directly, divert valves 372, 373 and 374 can divert the condensate from each stage to recycles or out of the system.

Each filtration stage removes part of the water in the ethanol product until the desired amount of water is removed, usually to produce an ethanol product of 99.5% by weight. The higher the concentration of vapor to be removed, water in this case, the higher the volume that will be removed. In the present example, suitably 60% to 70% of the water can be removed in the first stage. Also because of the selectivity of the membrane vs. volume of permeate removed, the amount of the organic solvent in the permeate condensate 327 suitably will be in the 100 PPM or less range. Suitably, this permeate can be removed directly from the system. The permeate from stage two 327 and stage three 347 will remove less amounts of permeate as the concentrations of the product reach higher purities. Suitably, 20% to 30% of the water is removed in stage two and 5% to 10% is removed in stage three and the percentage of ethanol in the condensed permeate is progressively higher. The design of the system will determine if it is necessary to recycle the permeate back to the distillation column 300 for recovery or send the water out as waste water. In stage four, 4% or less of the water is removed but it will have up to 0.5% or higher concentrations of ethanol. In most embodiments, this small amount of permeate 357 should be recycled back into the system through flow 371 for ethanol recovery.

Each membrane stage delivers higher and higher concentrations of ethanol in the condensate output of the stage. A portion of each or all stages can be recycled back to the still as reflux 368 to the distillation column 300. If a portion of the concentrate flow is to be recycled back to the distillation column 300 as reflux 368, control valves 381, 383, 385 and 389 will regulate the amount of flow from the stage to be recycled. When flow from each stage is selected, back pressure valves 382, 384 and 386 will provide that back pressure. The recycle flow 380 from all stages is routed through heat exchanger 365 and flow 366 to pressure relief valve 367. The temperature and pressure of the reflux 368 are suitably matched to the conditions required by the position of the reflux 368 to the distillation column 300.

While four filtration units are shown in the example, more or fewer filtration units can be used depending on the feed concentration and energy efficiency required.

The process and examples described above have eliminated energy conservation fluid piping, with the associated heat exchangers, possible in the implementation of the invention presented here. Many modifications of the described fluid piping for energy conservation are possible depending on the operating conditions selected for the process. Three such modifications are: 1) heat conservation can be achieved by transferring the heat energy of the purified concentrate to the incoming stream to feed the distillation column; 2) heat energy added at the pervaporation inter-stage heaters can be recovered from the vapor condenser at the overhead vapor outlet of the distillation column; and if one or more of the pervaporation stages is operated at temperatures higher than the distillation column's bottom heater (reboiler), part of that added heat could be derived from the concentrate product stream with an internal heat exchanger as part of the configuration of the distillation column's bottom heater (reboiler).

While the present invention has now been described and exemplified with some specificity, those skilled in the art will appreciate the various modifications, including variations, additions, and omissions that may be made in what has been described. Accordingly, it is intended that these modifications also be encompassed by the present invention and that the scope of the present invention be limited solely by the broadest interpretation that lawfully can be accorded the appended claims.

What is claimed is:

1. A method of separating ethanol from an ethanol/water mixture, the method comprising:
supplying an ethanol/water mixture through a primary feed to a fractional distillation column; the fractional distillation column producing a first ethanol enriched product, the first ethanol enriched product leaving the fractional distillation column through an overhead vapor outlet in the fractional distillation column;
introducing the first ethanol enriched product to a first filtration unit comprising a first input, a first selective membrane, a first concentrate output, and a first permeate output; the first ethanol enriched product being introduced to the first filtration unit through the first input; the first ethanol enriched product contacting the first selective membrane, a portion of the first ethanol enriched product passing through the first selective membrane, this portion is enriched in water and is a first water permeate, and a second portion of the first ethanol enriched liquid being further enriched in ethanol not passing through the first selective membrane, this portion being a first ethanol concentrate.

2. The method of claim 1 wherein the first ethanol concentrate has 60-70% less water than the ethanol/water mixture.

3. The method of claim 1 wherein the first ethanol enriched liquid is conditioned to a temperature of between about 200-400° F. and a pressure of between about 14-80 PSIG before passing through the first input.

4. The method of claim 1 wherein a portion of the first ethanol concentrate is reintroduced to the first filtration unit.

5. The method of claim 1 wherein the first ethanol concentrate is reintroduced to the fractional distillation column.

6. The method of claim 1 wherein the first ethanol enriched product is introduced into the first filtration unit in a liquid state.

7. The method of claim 1 wherein the first ethanol enriched product is introduced into the first filtration unit in a gaseous state.

8. The method of claim 1 wherein the first water permeate is introduced to a re-boiler operatively connected to the fractional distillation column, the re-boiler producing an ethanol enriched water permeate fraction which is introduced into the fractional distillation column, and a water enriched water permeate.

9. The method of claim 1 wherein the first ethanol concentrate is introduced to a second filtration unit comprising a second input, a second selective membrane, a second concentrate output, and a second permeate output; the first ethanol concentrate being introduced to the second filtration unit through the second input; the first ethanol concentrate contacting the second selective membrane, a portion of the first ethanol concentrate passing through the second selective membrane, this portion is enriched in water and is a second water permeate, and a second portion of the first ethanol concentrate not passing the second selective membrane, this portion being enriched in ethanol and is a second ethanol concentrate.

10. The method of claim 9 wherein the second ethanol concentrate has 20-30% less water than the first ethanol concentrate.

11. The method of claim 9 wherein the first ethanol concentrate is conditioned to a temperature of between about 200-400° F. and a pressure of between about 14-80 PSIG before passing through the second input.

12. The method of claim 9 wherein a portion of the second ethanol concentrate is reintroduced to the second filtration unit.

13. The method of claim 9 wherein the second ethanol concentrate is reintroduced to the fractional distillation column.

14. The method of claim 9 wherein the first ethanol concentrate present is introduced to the second filtration unit in a liquid state.

15. The method of claim 9 wherein the first ethanol concentrate is introduced to the second filtration unit in a gaseous state.

16. The method of claim 9 wherein the second water permeate is introduced to a re-boiler operatively connected to the fractional distillation column, the re-boiler producing an ethanol enriched water permeate fraction which is introduced into the fractional distillation column, and a water enriched water permeate.

17. The method of claim 9 wherein the second ethanol concentrate is introduced to a third filtration unit comprising a third input, a third selective membrane, a third concentrate output, and a third permeate output; the second ethanol concentrate being introduced to the third filtration unit through the third input; the second ethanol concentrate contacting the third selective membrane, a portion of the second ethanol concentrate passing through the third selective membrane, this portion is enriched in water and is a third water permeate, and a second portion of the second ethanol concentrate not passing the third selective membrane, this portion being enriched in ethanol and is a third ethanol concentrate.

18. The method of claim 17 wherein the third ethanol concentrate has 5-10% less water than the second ethanol concentrate.

19. The method of claim 17 wherein the second ethanol concentrate is conditioned to a temperature of between about 200-400° F. and a pressure of between about 14-80 PSIG before passing through the third input.

20. The method of claim 17 wherein a portion of the third ethanol concentrate is reintroduced to the third filtration unit.

21. The method of claim 17 wherein the third ethanol concentrate is reintroduced to the fractional distillation column.

22. The method of claim 17 wherein the second ethanol concentrate is introduced to the third filtration unit in a liquid state.

23. The method of claim 17 wherein the second ethanol concentrate is introduced to the third filtration unit in a gaseous state.

24. The method of claim 17 wherein the third water permeate is introduced to a re-boiler operatively connected to the fractional distillation column, the re-boiler producing an ethanol enriched water permeate fraction which is introduced into the fractional distillation column, and a water enriched water permeate.

25. The method of claim 17 wherein the third ethanol concentrate is introduced to a fourth filtration unit comprising a fourth input, a fourth selective membrane, a fourth concentrate output, and a fourth permeate output; the third ethanol concentrate being introduced to the fourth filtration unit through the fourth input; the third ethanol concentrate contacting the fourth selective membrane, a portion of the third ethanol concentrate passing through the fourth selective membrane, this portion is enriched in water and is a fourth water permeate, and a second portion of the third ethanol concentrate not passing the fourth selective membrane, this portion being enriched in ethanol and is a fourth ethanol concentrate.

26. The method of claim 25 wherein the fourth ethanol concentrate has 4% or less water than the third ethanol concentrate.

27. The method of claim 25 wherein the third ethanol concentrate is conditioned to a temperature of between about 200-400° F. and a pressure of between about 14-80 PSIG before passing through the fourth input.

28. The method of claim 25 wherein a portion of the fourth ethanol concentrate is reintroduced to the fourth filtration unit.

29. The method of claim 25 wherein the fourth ethanol concentrate is reintroduced to the fractional distillation column.

30. The method of claim 25 wherein the third ethanol concentrate is introduced to the fourth filtration unit in a liquid state.

31. The method of claim 25 wherein the third ethanol concentrate is introduced to the fourth filtration unit is in a gaseous state.

32. The method of claim 25 wherein the fourth water permeate is introduced to a re-boiler operatively connected to the fractional distillation column, the re-boiler producing an ethanol enriched water permeate fraction which is introduced into the fractional distillation column, and a water enriched water permeate.

33. A method of separating ethanol from an ethanol/water mixture, the method comprising:
supplying an ethanol/water mixture through a primary feed to a fractional distillation column; the fractional distillation column producing a first ethanol enriched product, the first ethanol enriched product leaving the fractional distillation column through an overhead vapor outlet in the fractional distillation column;
introducing the first ethanol enriched product to a first filtration unit comprising a first input, a first selective membrane, a first concentrate output, and a first permeate output; the first ethanol enriched product being introduced to the first filtration unit through the first input; the first ethanol enriched product contacting the first selective membrane, a portion of the first ethanol enriched product passing through the first selective membrane, this portion is enriched in water and is a first water permeate, and a second portion of the first ethanol enriched product being further enriched in ethanol not passing through the first selective membrane, this portion being a first ethanol concentrate;
introducing the first ethanol concentrate to a second filtration unit comprising a second input, a second selective membrane, a second concentrate output, and a second permeate output; the first ethanol concentrate being introduced to the second filtration unit through the second input; the first ethanol concentrate contacting the second selective membrane, a portion of the first ethanol concentrate passing through the second selective membrane, this portion is enriched in water and is a second water permeate, and a second portion of the first ethanol concentrate not passing the second selective membrane, this portion being enriched in ethanol and is a second ethanol concentrate;
introducing the second ethanol concentrate to a third filtration unit comprising a third input, a third selective membrane, a third concentrate output, and a third permeate output; the second ethanol concentrate being introduced to the third filtration unit through the third input; the second ethanol concentrate contacting the third selective membrane, a portion of the second ethanol concentrate passing through the third selective membrane, this portion is enriched in water and is a third water permeate, and a second portion of the second ethanol concentrate not passing the third selective membrane, this portion being enriched in ethanol and is a third ethanol concentrate.

34. The method of claim 33 wherein portions of the first, second and third ethanol concentrates are reintroduced to the fractional distillation column.

35. The method of claim 33 wherein a portion of the first ethanol concentrate is reintroduced to the first filtration unit; a portion of the second ethanol concentrate is reintroduced to the second filtration unit; and a portion of the third ethanol concentrate is reintroduced to the third filtration unit.

36. The method of claim 33 wherein the first, second and third water permeates are introduced to a re-boiler operatively connected to the fractional distillation column, the re-boiler producing an ethanol enriched water permeate fraction which is introduced into the fractional distillation column, and a water enriched water permeate.
